(19) United States
(12) Patent Application Publication
(10) Pub. No.: US 2020/0022920 A1
(43) Pub. Date: Jan. 23, 2020

(54) Rupture-Stable Gelatin Filled Capsules and Manufacturing Thereof

(71) Applicant: General Nutrition Investment Company, Wilmington, DE (US)

(72) Inventors: Binh Nguyen-Davis, Taylors, SC (US); Jowell G. Bolivar, Simpsonville, SC (US); Jeffrey T. Taylor, Simpsonville, SC (US)

(21) Appl. No.: 16/514,328

(22) Filed: Jul. 17, 2019

Related U.S. Application Data
(60) Provisional application No. 62/699,324, filed on Jul. 17, 2018.

(51) Int. Cl. A61K 9/48; A61J 3/07

(52) U.S. Cl. CPC A61K 9/4825; A61J 3/07; A61K 9/4875; A61K 9/4858

(57) ABSTRACT

The present invention relates to a rupture-stable gelatin filled capsule, produced from xylose cross-linked gel masses. The xylose cross-linked masses are made from a 0.01%-2% xylose solution combined with gelatin masses. The gelatin masses may be produced from gelatin of bovine, pork, fish, chicken, duck, or other poultry. The xylose solutions and gelatin masses are then mixed at temperatures from 135-200° F. and yield viscosities from 10,000-16,000 cps, creating a capsule which can be resistant to rupture in gastric digestives, but which will then rupture after a time period within the intestinal tract.

### CROSS REFERENCE TO RELATED APPLICATION

This application claims priority to U.S. Provisional Patent Application No. 62/699,324, filed on Jul. 17, 2018, the disclosure of which is hereby incorporated in its entirety by reference.

### BACKGROUND OF THE INVENTION

### Field of the Invention

The present invention relates to gelatins. In particular, but not exclusively, the invention relates to gelatin filled capsules for timed release in a subject, and manufacturing thereof.

### Description of Related Art

Gelatin is a naturally occurring protein collagen derived from bovine bones and hides, pork skin, and other animal sources such as chicken, duck, and fish. It is widely used in the pharmaceutical and food industries. In the nutraceutical industries, gelatin is used as protective encapsulants in hard-, and soft-capsule oral delivery systems. Soft capsules are designed to deliver liquid and paste type actives, while hard capsules are the preferred delivery format for dry blend powder mixtures. Gelatin is also used to enrode tablets as coating polymer to protect the tablets from oxygen, moisture, and light.

Gelatin is recovered from animal collagen through hydrolytic reactions. Gelatin is processed and generally classified as Type A gelatin or Type B gelatin. Type A gelatin comes from pork skins and is processed by washing the pork skin with cold water, and soaking washed pork skin in cold dilute mineral acid for several hours until the gelatin reaches maximum swelling capacities. On the other hand, Type B gelatin is obtained from ossein and bovine hide, and is processed for several hours with alkali (usually lime) and water at ambient temperatures. Liming takes usually 8-12 weeks depending on the starting materials and lime used. Ossein usually requires more liming time, and could take from 5-20 weeks longer. In the final processing stages, Type B gelatin is washed with cold water and the resulting product undergoes hot-water extractions to recover soluble gelatin.

Gelatin is generally composed of amino acids in a typical profile, such as glycine 25.5%, proline 18%, hydroxyproline 14.1%, glutamic acid 11.4%, alanine 8.5%, arginine 8.5%, aspartic acid 6.6%, lysine 4.1%, leucine 3.2%, valine 2.5%, phenylalanine 2.2%, threonine 1.9%, isoleucine 1.4%, methionine 1.0%, histidine 0.8%, tyrosine 0.5%, serine 0.4%, and cysteine 0.1%.

Because of these amino acid functional moieties in gelatin, several inventions have been published in making derivatives of gelatins. U.S. Pat. No. 2,525,753 discloses a chemical derivatization method of gelatin forming distinctive properties from the reactions of gelatin with one or more of the following anhydrides: phthalic, maleic, succinic, and their halogen and sulfo-substituted derivatives.

U.S. Pat. No. 2,816,099 discloses a process for making protein isocyanic acid derivatives. This particular invention concerns the treatment of water sensitive proteinaceous materials, such as gelatin, in the presence of isocyanic acid whereby substituted proteinaceous materials, including gelatin, are obtained.

U.S. Pat. No. 3,108,995 discloses a method of modifying type A gelatin, wherein type A gelatin at a pH range from 3 to 8.5, and temperature range of 25 to 90° C., was modified using sufficient polycarboxylic acid compounds selected from the group consisting of succinic, maleic, phthalic, citraconic, itaconic and aconitic anhydrides, and succinyl and fumaryl chlorides and mixtures. The modified type A gelatin from these processes have lowered the isoelectric point and are within the pH range of 4.0 to 5.5.

U.S. Pat. No. 7,485,323 describes chemical modification processes for the pharmaceutical grades of either type A or B gelatin. The gelatin in this work underwent proteolytic hydrolyses reactions, and the resulting gelatin hydrolysates were reacted to 2-(4-Dimethylcarbamoyl-pyridino)-ethane-1-sulfonate. This invention allowed for controlled gelatin manufacturing processes for improved stability and dissolution of gelatin capsules. This invention is called GELITA®RXL.

Recently, U.S. Pat. No. 9,611,405 describes the use of nano-particles from inorganic materials including titanium dioxide, aluminum oxide, silicon dioxide, zirconium dioxide, indium tin oxide, zinc oxide, zinc sulfide, molybdenum sulfide or silver; to make a stable gelatin dispersion for making films, foils, or coatings.

Gelatin exhibits poor mechanical properties, especially when exposed to wet and/or humid conditions, and the high water sensitivity compromises its broader applications. To improve the thermal, mechanical, and water-resistance capabilities of gelatin, chemical cross-linking modifications have been successfully performed through gelatin amino acid moieties including amine, carboxyl, or hydroxyl groups. Different chemical and natural systems such as glyoxal, epoxides, isocyanates, carbodiimides, formaldehyde, tannin,ferulic acid, glyceraldehyde, genepin, and even enzymes such as transglutaminase had been used to cross-link gelatin. Recently, U.S. Pat. No. 9,295,751 discloses an invention using transglutaminase cross-linked with gelatin, and the inventors claimed medical glue applications in human or animal bodies.

Xylose has also been used to chemically cross-link gelatin. It is a major component of plants and also one of the most abundant carbohydrates on earth, second only to glucose. Xylose can also be converted into xylulose and further into furfural under mild conditions—furfural is a biomass-derived platform of great chemical interest. Xylose can also be processed through microbial conversions using Saccharomyces cerevisiae, for fuels and chemical applications.

EP 0,240,581 discloses a soft capsule gelatin process formulation wherein 2 parts of xylose were added to 100 parts gelatin mass. The resulting dry capsule shell could swell in artificial body fluids allowing active substances to diffuse slowly. The inventors did not make any specific claims for xylose as the exact and only cross-linker molecule, but listed physiologically and toxicologically acceptable aldehydes having at least 4 carbon atoms, namely terpenes, cinnamaldehyde, and aldoses or mixtures of aldoses.

U.S. Pat. No. 7,264,824 discloses spraying techniques to cross-link hardened gelatin with xylose solutions. In this work, heat treatments were applied to initiate cross-linking processes between the aldehyde functionalities in xylose molecules and gelatin. The resulting xylose cross-linked gelatin capsules inhibited peroxidation processes of fatty acids including perilla oils, as exhibited by low peroxide values after 12 month studies.

The chemistry of gelatin cross-linking may occur through one or several reactions. This could be (a) a series of aldol-type condensation reactions to produce a cross-linked product having pyridinium rings; (b) reactions of a lysyl ε-amino group with an aldehyde; (c) formation of an imine arising from the reactions between the ε-amino groups of lysine molecules and aldehydes of xylose—which results to aminal formations; and (d) reactions between aldehyde functional group sugars with the ε-amino groups in gelatin forming an imine intermediate and ketose sugars.

### SUMMARY OF THE INVENTION

The invention relates to rupture-stable gelatin filled capsules, produced by using two mechanisms in which xylose cross-links with gelatin. First is the cross-linking process that takes place between sugars and gelatin, forming an imine intermediate and ketose sugars. Another is Schiff base adduct reactions between xylose aldehyde moieties and ε-amine groups of lysine molecules in gelatin. The latter is also a Maillard type reaction which causes the browning effect in cross-linked xylose and gelatin invented here.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 shows a schematic of cross-linked gelatin according to the present invention.

FIG. 2 shows a different schematic of cross-linked gelatin according to the present invention.

FIG. 3 is a colored depiction of the color differences of xylose-treated gelatin masses according to the present invention.

FIG. 4 shows a side by side comparison of prior art capsules and the rupture-stable gelatin filled capsules according to the present invention.

FIG. 5 is a colored photo diagramming the UV absorption of xylose-treated gelatin masses according to the present invention.

FIG. 6 shows a comparison of prior art capsules and the rupture-stable gelatin filled capsules after set time periods in a digestive medium according to the present invention.

### DESCRIPTION OF THE INVENTION

Non-limiting embodiments or aspects of the present invention are directed to gelatins, methods, and products for manufacturing rupture-stable soft gelatin filled capsules.

The present invention involves gelatin masses. According to one non-limiting preferred embodiment, gelatin masses were prepared using glycerin, gelatin, and water in differing ratios, preferably 1:2.5:2. Varying temperatures were also used in preparation, from approximately 135-200° F. Upon total melting of the gelatin, it was cooled down to approximately 135-150° F. The gelatin masses yielded viscosities between 12,000 and 16,000 cps.

To initiate cross-linking processes, 0.1% to 5% by weight aqueous solutions of D-xylose were added to the hot gelatin mixtures. This was added approximately 1-2 hours prior to encapsulation. The resulting xylose-gelatin mixtures were mixed for at least 15 minutes at temperatures above 140° F. Water was added in as necessary to keep viscosities between 10,000 and 16,000 cps.

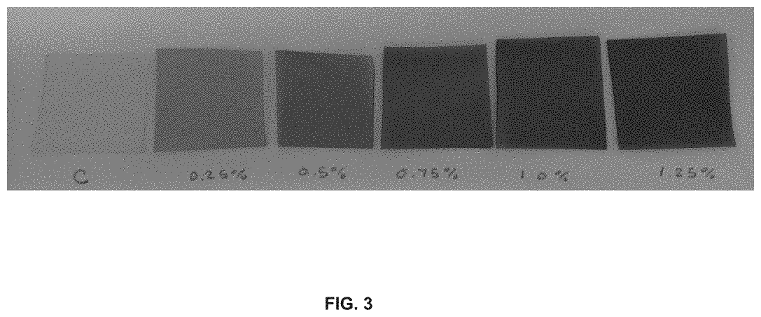

After approximately 12 hours, the xylose-gelatin masses start cross-linking. This is recognized by color changes of FIG. 3. In FIG. 3, non-xylose gelatin masses (labeled as control, C), were clear. Gelatin masses with increased xylose treatment ranged in color from clear to amber, with the xylose ranging from −0.25% to 1.25% weight xylose. The initial results display successful degrees of cross-linking processes using xylose concentrations stepping in 0.25% increments from 0.25% to 1.25%.

It is noted that the most efficient cross-linking processes were obtained using xylose levels of 0.75% to 1.25% concentrations, as displayed by the golden brown colors. The golden brown colors of the cross-linked gelatins differ from colors of enteric-coated soft gelatin capsules. Enteric-coated softgels have a frosted finish with white spots or specks, due to spray-coating processes used. The present invention utilizes the xylose-gelatin cross-linking process which allows for a homogeneous and natural pure brown look of softgels, while using only machineries or equipments commonly used in the manufacturing of soft gelatin capsules.

The process described above eliminates expensive and laborious manufacturing steps as compared to using spray coating technologies for creating enteric-coated softgels.

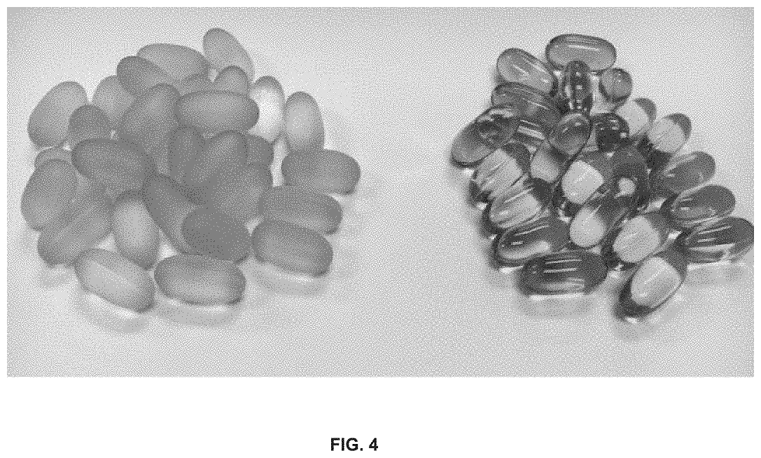

FIG. 4 further displays comparisons between the “natural” golden brown colors of rupture-stable softgel-filled capsules invented herein, as opposed to the frosted appearance of enteric-coated softgels.

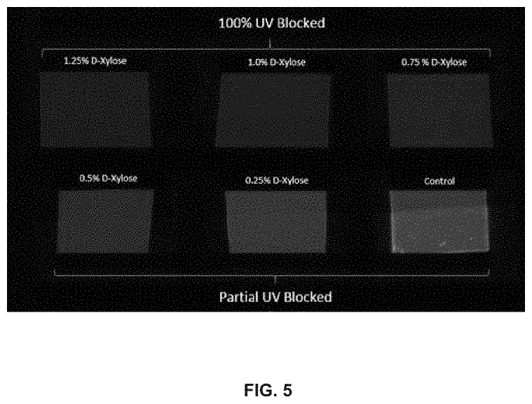

Ultraviolet spectroscopy was also used in the present invention to assess successful cross-linking processes between xylose and gelatin masses. FIG. 5 demonstrates photomicrographs of gelatin masses containing different levels of xylose. Gelatin masses cross-linked with at least 0.25% by weight of xylose showed degrees of resistance towards UV light absorption at 366 nm, and exhibited partial dull backgrounds. Complete resistance to UV light absorptions, demonstrated by dull backgrounds, were observed at 0.75% to 1.25% by weight of xylose addition. This suggested that UV light was completely blocked from penetrating xylose cross-linked gel masses and could offer protection for any fill materials which are prone to light oxidation including, but not limited to, polyunsaturated fatty acids derived from fish and vegetable oils.

Gelatin masses without xylose cross-linking displayed illumination, or “glow”, regions, when presented with the above-identified test (i.e., exposed under UV light at 366 nm wavelength). This confirmed that UV light was absorbed into the gelatin masses and, therefore, could reach light sensitive fill materials such as the polyunsaturated fatty acids. This would deteriorate the fill materials over time. No prior inventions have established UV spectroscopy to assess cross-linking between xylose and gelatin matrices.

Successful cross-linking processes between xylose and gelatin, achieved through normal manufacturing processes, resulted in rupture-stable cross-linked gel masses of the present invention. The rupture-stable cross-linked capsules are used to encapsulate polyunsaturated fatty acids. In one preferred non-limiting embodiment, the polyunsaturated fatty acids were derived from omega-3 fish oils.

A softgel encapsulation machine was utilized to form xylose and gelatin cross-linked capsules containing a polyunsaturated fatty acid. Here, the gelatin and xylose mixture is fed into the spreader box, which spreads the molten gelatin mixture onto a rotating drum to form a ribbon of gelatin/xylose. The gelatin/xylose ribbon is fed into a die roll which forms a capsule, while the injection wedge simultaneously injects the active ingredient e.g., polyunsaturated fatty acids into the capsule. The injection wedge is heated to facilitate capsule sealing after dispensing the active ingredient. Spreader box temperatures of 130-160° F., and wedge temperatures of 95-130° F. were adjusted proportional to the age of cross-linked gel masses. Cross-linked gelatin capsules were set to cure for approximately 1-3 weeks, depending on the amount of xylose concentration used to cross-link the gel masses. Capsules which were allotted cure times of 3 weeks achieved both gastric and intestinal disintegration requirements following USP 2040 Disintegration and Dissolution of Dietary Supplements.

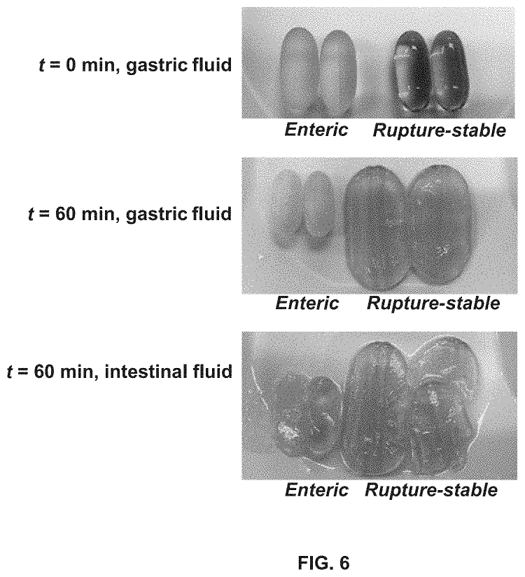

At 8 weeks old, the capsules were still able to pass USP 2040 requirements, i.e., the capsules did not disintegrate in simulated gastric fluid for 60 minutes, and would rupture in 55 minutes in simulated intestinal fluid. FIG. 6 is a comparison between enteric-coated filled softgels and cross-linked filled softgels at different times within the test.

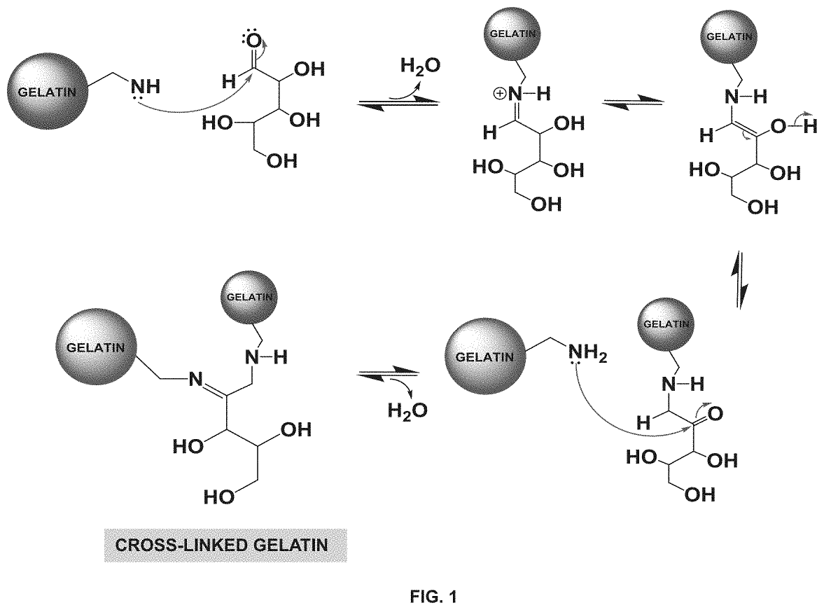

The cross-linking reaction of FIG. 1 involves the reaction of a secondary amine group of gelatin with the aldehyde group of xylose. The reaction proceeds to form an imine group, wherein the nitrogen atom of gelatin is double bonded to xylose. A rearrangement of the imine intermediate allows for the formation of a ketose sugar, wherein the neighboring hydroxyl group of xylose is converted to a ketone. This ketone can then react with a primary amine group of gelatin (ε-amine of lysine) to form an additional gelatin-xylose cross-link.

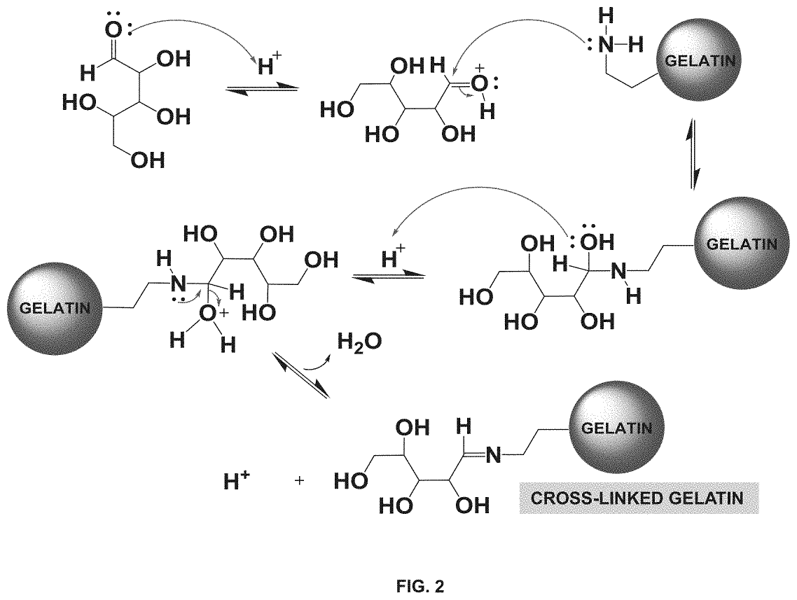

The proposed cross-linking reaction of FIG. 2 is that of a Maillard type reaction. The Maillard reaction involves a reaction between sugars and amino acids in the presence of heat. The resulting products of these reactions cause a browning effect and explains the color change observed within the prepared cross-linked gelatin. Here, the xylose reacts with the ε-amine group of lysine within the gelatin backbone. This reaction forms a Shiff base and cross-links the gelatin molecule to the xylose through the formation of a carbon-nitrogen double bond.

The invention claimed is:

1. A rupture-stable gelatin filled capsule, comprising xylose cross-linked gel masses wherein the xylose cross-linked masses are comprised of 0.01%-2% xylose solutions and gelatin masses, and wherein the xylose solutions and gelatin masses are mixed at temperatures from 135-200° F. and yield viscosities from 10,000-16,000 cps.

2. The rupture-stable gelatin filled capsule as in claim 1, comprised of 075%-1.25% xylose solutions.

3. The rupture-stable gelatin filled capsule of claim 1, wherein the mixing temperature is within 150-200° F.

4. The rupture-stable gelatin filled capsule of claim 1, wherein the xylose solution is prepared from xylose sugars, such as D-xylose, L-xylose, or DL-xylose.

5. The rupture-stable gelatin filled capsule of claim 1, wherein the gelatin masses comprise 8%-27% glycerin, 21-66% gelatin, and 17-53% water.

6. The rupture-stable gelatin filled capsule of claim 1, wherein the gelatin masses comprise 17.5% glycerin, 43.5% gelatin, and 35% water.

7. The rupture-stable gelatin filled capsule of claim 1, wherein the gelatin masses are of bovine, pork, fish, chicken, duck, or other poultry origin.

8. The rupture-stable gelatin filled capsule of claim 1, wherein the capsule contains nutraceutical ingredients.

9. The rupture-stable gelatin filled capsule of claim 1, wherein the capsule is a clear golden brown color.

10. The rupture-stable gelatin filled capsule of claim 9, wherein the nutraceutical ingredients comprise polyunsaturated fatty acids, derived from omega 3-oils of fish, hill, green lipped mussel, algae, and/or plants.

11. The rupture-stable gelatin filled capsule of claim 9, further comprising lipid-based nutraceutical ingredients as filler materials, such as saw palmetto.

12. The rupture-stable gelatin filled capsule of claim 9, containing nutraceutical ingredients such as garlic, valerian, milk thistle, N-acetyl cysteine, S-adenosylmethionine, quercetin, pyrroloquinoline quinone (PQQ), alpha-lipoic acid, thiamine hydrochloride, thiamine mononitrate, Vitamin C, Vitamin K1, Vitamin K2, Vitamin K3, Vitamin D2, Vitamin D3, Vitamin A and beta-carotene, lycopene, Vitamin E TPGS, Vitamin E forms including mixed tocopherols, chamomile, turmeric, ginger, dill weed, mineral forms of iron, copper, and manganese to be used as softgel filler materials.

13. The rupture-stable gelatin filled capsule of claim 1, wherein the capsule withstands disintegration for 60 minutes after placement in simulated gastric fluid.

14. The rupture-stable gelatin filled capsule of claim 13, wherein the capsule disintegrates upon placement into simulated intestinal fluid within 60 minutes.

15. A method of producing a rupture-stable gelatin filled capsule, comprising the steps of:
a) mixing glycerin, gelatin, and water in ratios of 1:2.5:2 by weight;
b) controlling the temperature of the mixture with a temperature controlled melter to approximately 150-200° F.;
c) heating the mixture until the gelatin has completely melted;
d) allowing the mixture to cool down to temperatures between 135-150° F.;
e) adding 0.1%-5% by weight of aqueous D-xylose to the hot gelatin mixtures;
f) mixing the resulting xylose-gelatin mixture for at least 15 minutes at temperatures above 140° F.;
g) adding water to the xylose-gelatin mixture to maintain viscosities of 10,000-16,000 cps; and
h) encapsulating nutrapharmaceuticals within the xylose-gelatin mixture.
